(19) United States
(12) Patent Application Publication
(10) Pub. No.: US 2018/0179568 A1
(43) Pub. Date: Jun. 28, 2018

(54) Method of Pretreating Lignocellulose using Solvent with Little Water Footprint

(71) Applicant: Institute of Nuclear Energy Research, Atomic Energy Council, Executive Yuan, R.O.C., Taoyuan City (TW)

(72) Inventors: Fong-Yu Yen, Taoyuan (TW); Hsin-Hung Chen, New Taipei City (TW); Gia-Luen Guo, New Taipei City (TW); Ming-Feng Jang, New Taipei City (TW); Wen-Hua Chen, New Taipei City (TW)

(21) Appl. No.: 15/390,759

(22) Filed: Dec. 27, 2016

(51) Int. Cl. C12P 19/14; C12P 19/04; C08L 97/02

(52) U.S. Cl. CPC C12P 19/14; C12P 2201/00; C08L 97/02; C12P 19/04

(57) ABSTRACT

Lignocellulose is pretreated using solvent with little water footprint. Both a solvent and water are used for saccharification and separating the components of lignocellulose. By adding a small amount of an inorganic acid, lignin is dissolved and hemicellulose is hydrolyzed under appropriate reaction temperature and pressure. Both are dissolved to form liquid phase with solid cellulose residue left. The relative volatility difference between the solvent and water is used to selectively remove more of the solvent. More particularly, drying with air-suction at a relatively low temperature helps remove and recycle the solvent from the cellulose solid residue. Thus, water consumption and the subsequent wastewater treatment can be significantly reduced with little water footprint while achieving the original desired efficiency of enzymatic hydrolysis. The concentration of the solution consisting of the solvent of lignin and the hydrolyzate of hemicellulose can be reduced to precipitate solid lignin through evaporation or dilution.

### TECHNICAL FIELD OF THE INVENTION

The present invention relates to pretreating lignocellulose; more particularly, to improving removing solvent of lignocellulose, where water consumption is reduced with little water footprint.

### DESCRIPTION OF THE RELATED ARTS

Wastes from agriculture and forestry are so-called lignocellulose source materials, which consist of three main components—namely cellulose, hemicellulose, and lignin. Therein, cellulose and hemicellulose are mainly composed of chained fermentable monosaccharides like six-carbon glucose, five-carbon xylose, etc. Lignin is composed of three different phenylpropanoid monomers. From the viewpoint of biological resource, they have use values. However, since biomass is formed by itself into a complex structure with a delivery mechanism for self-protection without losing water and sugar on being damaged by animal or bacteria, the biggest challenge for refining valuable products from lignocellulose is on how to overcome the recalcitrant structure.

To process substance conversion in lignocellulose has two main ways, namely sugar platform and thermochemical platform. Saccharification is the key process in the sugar platform. From the viewpoint of saccharification, owing to the structural strength difference between cellulose and hemicellulose, well-known available saccharification mainly chooses hemicellulose at first to be hydrolyzed into xylose, which is a fermentable monosaccharide; and, then, tends to use enzymatic hydrolysis to convert cellulose into glucose. Thus, the whole saccharification is completed.

For the source material of lignocellulose, diluted acid is commonly used for pretreatment. The hemicellulose in the source material can be hydrolyzed into fermentable monosaccharide at first while the solid structure of the residue mainly consisting of cellulose and lignin is effective destructed for achieving better efficiency of enzymatic hydrolysis of cellulose.

As compared to another common treatment using a base, this method can solve the shortcoming of a lot of waste solution generated. However, the complex matrix composed of lignin and hemicellulose in lignocellulose would make enzyme hard to approach the activation position of cellulose. For a source material having a richer content of lignin, the complex matrix also adsorbs enzyme so that not only the structures of cellulose and lignin are even more difficult to be destructed, but also the adsorption and inhibition of the enzyme would significantly reduce the efficiency of the enzymatic hydrolysis owing to the existence of lignin. The so-called organsolv pretreatment usually uses water and a solvent for saccharification of lignocellulose. By adding a small amount of an inorganic acid, lignin can be dissolved and hemicellulose can be hydrolyzed under appropriate temperature and pressure while a relatively high purity of cellulose solid residue can be left. Thus, the enzymatic hydrolysis of cellulose can be processed with a lower dose of enzyme. Thus, the enzymatic inhibition to the source material containing a higher content of lignin can be effectively solved; meanwhile, high-value high-purity lignin, xylose or their derivatives can be separated out. However, because the residual solvent in the cellulose solid residue would also significantly inhibit the enzymatic hydrolysis in this method, the solvent must be removed and recycled before processing the enzymatic hydrolysis. The prior art usually uses water-washing to remove the solvent. In the process, a lot of water is required for multiple washing of the cellulose solid residue to remove most of the solvent. Then, enzyme is added to hydrolyze cellulose into glucose as a material for subsequent fermentation for producing biofuels or biobased-chemicals. Yet, the subsequent issues regarding recycling, waste water treatment, etc. generate concerns on cost, environmental protection, and water acquisition and configuration. Hence, the prior arts do not fulfill all users' requests on actual use.

### SUMMARY OF THE INVENTION

The main purpose of the present invention is to improve removing the solvent of lignocellulose, where water consumption is reduced with little water footprint.

Another purpose of the present invention is to use the volatility difference between the solvent and water, especially under a relatively low temperature, to remove and recycle the solvent of the cellulose solid residue through drying with air-suction, where water consumption during production and subsequent wastewater treatment capacity are significantly reduced with little water footprint and the original hydrolysis efficiency of the pretreatment using the solvent is achieved.

Another purpose of the present invention is to reduce the solvent concentration of the solution containing lignin and hemicellulose hydrolyzates by way of evaporation or dilution to precipitate solid lignin for effectively separating three main components of lignocellulose for taking full advantage and wide applications of the lignocellulose.

Another purpose of the present invention is to separate high-purity products including lignin, cellulose, and hemicellulose for high-value applications, where the present invention is applicable to high-price source material of lignocellulose containing high content of lignin; and the present invention effectively reduces water consumption during production for helping the plant domain lack of water resource.

To reach the above purposes, the present invention is a method of pretreating solvent of lignocellulose with little water footprint, comprising steps of: (a) providing a source material of lignocellulose, where the source material of lignocellulose comprises biomass of cellulose, hemicellulose, and lignin; and has a lignin content more than 10 percents (%); (b) cutting the source material of lignocellulose to desired sizes; (c) uniformly mixing the source material of lignocellulose with a solvent through blending to process a high-temperature high-pressure reaction in a reactor, where the solvent comprises an alcohol, water and an acid; and the high-temperature high-pressure reaction is processed at a temperature of 120˜160 celsius degrees (° C.) for 20˜120 minutes; (d) separating solid content and liquid content in the reactor to obtain a cellulose solid residue having a high cellulose content and a hydrolyzate of lignin and xylose; (e) processing the hydrolyzate to precipitate lignin and separate xylose through a method selected from a group consisting of evaporation and solvent-concentration dilution; (f) recycling the solvent; (g) removing the solvent contained in the cellulose solid residue through low-temperature drying and recycling the solvent by looping back to step (f), where the low-temperature drying is coordinated with air-suction and is processed at a temperature of 10˜50° C. under a pressure of 10˜150 millibars (mbar); and (h) processing the cellulose solid through enzymatic hydrolysis with a cellulase, where the hydrolysis has a saccharification rate up to 80%. Accordingly, a novel method of pretreating solvent of lignocellulose with little water footprint is obtained.

### BRIEF DESCRIPTION OF THE DRAWINGS

The present invention will be better understood from the following detailed description of the preferred embodiment according to the present invention, taken in conjunction with the accompanying drawings, in which

FIG. 1 is the flow view showing the preferred embodiment according to the present invention;

FIG. 2 is the view showing the cellulose solid residue and the precipitated lignin;

FIG. 3 is the view showing the hydrolysis efficiencies with solvent removed through different ways;

FIG. 4 is the view showing the volatility trend of ethanol and water at different temperatures; and

FIG. 5 is the flow view showing the second state-of-use of the preferred embodiment.

### DESCRIPTION OF THE PREFERRED EMBODIMENT

The following description of the preferred embodiment is provided to understand the features and the structures of the present invention.

Based on a sugar platform for biological fermentation conversion, the present invention explores the key process of saccharification. Namely, an appropriate method is used to efficiently convert cellulose and hemicellulose of lignocellulose into fermentable monosaccharides for subsequent fermentation or other applications. Therefore, the present invention is to improve the process of solvent removal. The solvent is removed and recycled for reducing water consumption and the capacity of subsequent waste water treatment.

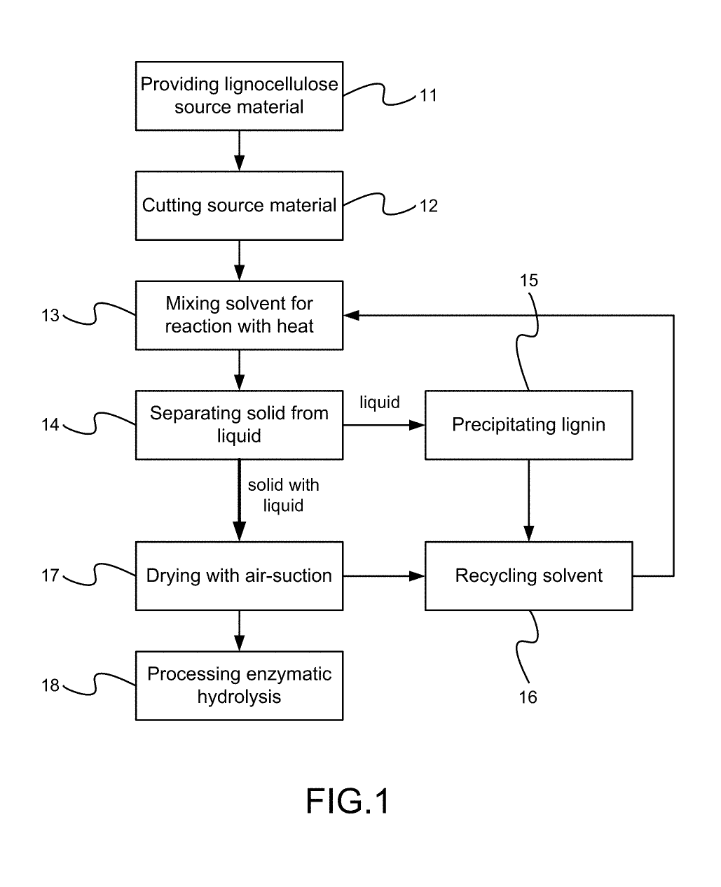

Please refer to FIG. 1, which is a flow view showing a preferred embodiment according to the present invention. As shown in the figure, the present invention is a method of pretreating lignocellulose using solvent with little water footprint, comprising the following steps:

(a) Providing lignocellulose source material 11: A source material of lignocellulose is provided. The source material of lignocellulose comprises biomass of cellulose, hemicellulose, and lignin; and has a lignin content more than 10 percents (%). For example, a non-grain lignocellulosic raw material of wood chips or bagasse can be provided.

(b) Cutting source material 12: The source material of lignocellulose is cut to desired sizes between 1 and 2 centimeters.

(c) Mixing solvent for reaction with heat 13: An appropriate ratio of the cut source material of lignocellulose is uniformly mixed with a solvent through blending to process high-temperature high-pressure reactions. Therein, the reactions are processed at a temperature of 120˜160 celsius degrees (° C.) for a time of 20˜120 minutes; the solvent comprises water, an alcohol, and an acid; the alcohol is methanol, ethanol, isopropanol, n-butanol or isobutanol; the acid is an inorganic acid or an organic acid; and, the inorganic acid is sulfuric acid, hydrochloric acid, or acetic acid. As desirably, the concentration of the alcohol can be adjusted within 40˜75%, that of water within 25˜60%, and that of the acid within 0.5˜3%; and a weight ratio of the solvent to the source material of lignocellulose is adjusted within 4˜12.

(d) Separating solid from liquid 14: When step (c) is finished, solid content and liquid content in the reactor are separated to obtain a cellulose solid residue and a hydrolyzate where the cellulose solid residue has a high content of cellulose and the hydrolyzate contains lignin and xylose.

(e) Precipitating lignin 15: By evaporating the hydrolyzate or diluting the concentration of the solvent, lignin is precipitated to be separated from xylose. Therein, for diluting the concentration of the solvent, a solvent different from alcohol, like water, is added.

(f) Recycling solvent 16: When step (e) is finished, the solvent is recycled.

(g) Drying with air-suction 17: The solvent contained in the cellulose solid residue is mainly removed through low-temperature drying and the solvent is recycled by looping back to step (f). Therein, the low-temperature drying is coordinated with air-suction and is processed at a temperature of 10˜50° C. under a pressure of 10˜150 millibars (mbar) for a time of 0.5˜12 hours.

(h) Processing enzymatic hydrolysis 18: The cellulose solid residue dried with air-suction is processed through enzymatic hydrolysis with a 10˜30 FPU/g cellulose. The reaction of enzymatic hydrolysis is processed at a temperature of 45˜52° C. for a time of 24˜72 hours to obtain a saccharification rate up to 80%.

Thus, a novel method of pretreat solvent of lignocellulose with little water footprint is obtained.

### [State-of-Use 1]

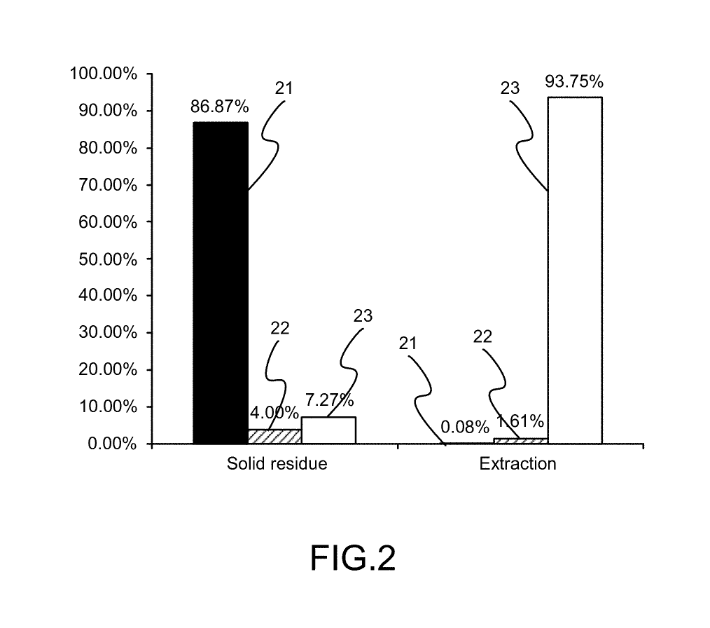

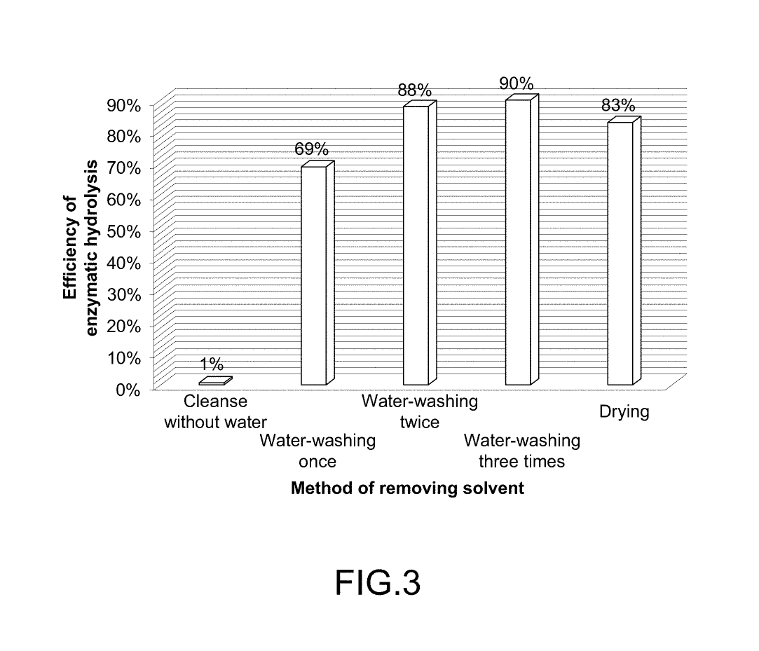

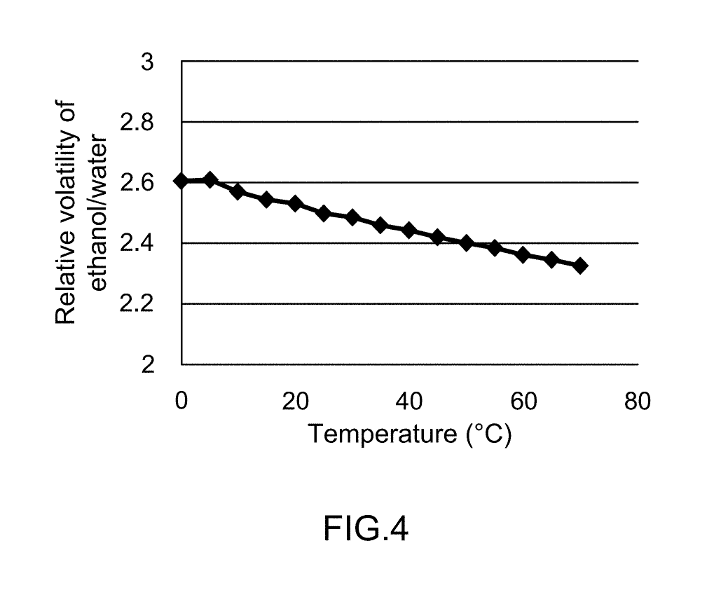

Please refer to FIG. 2˜FIG. 4, which are a view showing cellulose solid residue and precipitated lignin; a view showing hydrolysis efficiencies with solvent removed through different ways; and a view showing a volatility trend of ethanol and water at different temperatures. As shown in the figures, a first state-of-use according to the present embodiment uses wood chips as a source material of lignocellulose, where the source material is cut into sizes of 1˜1.5 centimeters (cm) to be uniformly mixed with 30˜50 percents (%) of water and 50˜70% of an alcohol-based solvent while 1˜2% of sulfuric acid is added. A solid/liquid phase is formed by a weight ratio of 10 of the solvent to the source material of lignocellulose for processing reaction in a reactor at a temperature of 135˜160° C. for 50˜80 minutes. In FIG. 2, cellulose contents 21, hemicellulose contents 22, and lignin contents 23 are shown. After finishing the reaction and separating solid from liquid, more than 90% of lignin is removed. The cellulose content in solid is more than 86%; and, after precipitating lignin from the hydrolyzate, the lignin purity is higher than 93%. After the reaction, the solid residue is cleansed without water or with water of the same volume once, twice, three times, and dried with air-suction. Then, a dose of 15 FPU/g cellulose of a hydrolyzing enzyme is used to process reaction at a temperature of 50° C. for 72 hours. As shown in FIG. 3, saccharification efficiencies of 1%, 69%, 88%, 90%, and 83% are obtained, respectively. Therein, the drying with air-suction is processed at a temperature below 30° C. under a pressure below 100 mbar. With an ethanol solvent used, a relative volatility of the solvent to water is about 2.5 to help remove the solvent; and, after drying the solid residue, the residual amount of the solvent can be less than 3%, as shown in FIG. 4. It is noticeable that the condition for drying with air-suction can be further optimized to expectedly improve the efficiency of saccharification.

### [State-of-Use 2]

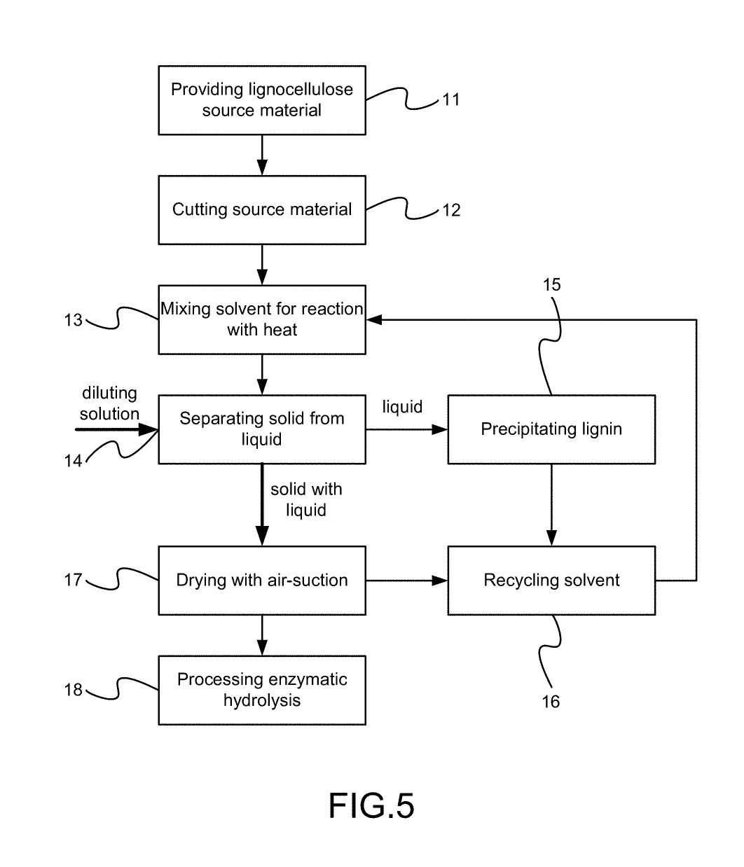

Please further refer to FIG. 5, which is a flow view showing a second state-of-use according to the preferred embodiment. As shown in the figure, the present invention improves the process of lignin precipitation. A small amount of diluent is added to enhance lignin precipitation during separating solid from liquid. Then, the remaining solvent in the solid residue is removed through drying with air-suction. The diluent used here is water, a solvent (e.g. an alcohol), an acid or a combination thereof.

The present invention provides a method to help remove the lignocellulosic solvent for reducing water consumption with little water footprint. By mixing lignocellulose with water and a solvent having a low boiling point, lignin is dissolved and hemicellulose is hydrolyzed under appropriate temperature and pressure after being added with a small amount of an inorganic acid. After being dissolved into a liquid phase, the cellulose solid residue is left over. The conventional method uses a plenty of water to wash cellulose solid residue for removing most of the residual solvent and adds an enzyme to hydrolyze cellulose into glucose, which is used as a material for producing biofuels or biomass chemicals through subsequent fermentation. The present invention uses the volatility difference between water and the solvent, especially under a relatively low temperature, to remove and recycle the solvent of the cellulose solid residue through drying with air-suction. Thus, water consumption during production and subsequent wastewater treatment capacity are significantly reduced with little water footprint while the original hydrolysis efficiency of the pretreatment using the solvent is achieved. The solvent concentration of the solution containing the hydrolyzate of lignin and hemicellulose can be reduced by way of evaporation or dilution to precipitate solid lignin for effectively separating three main components of lignocellulose for taking full advantage and wide applications of the lignocellulose. If the pretreatment proposed by the present invention uses a mixed solution having an appropriate ratio of alcohol to water with a suitable inorganic acid added, the lignin removal rate can be greater than 90%, the purity of glucan of the cellulose solid residue can be greater than 85%, and the purity of the precipitated lignin solid can be greater than 90%. If the solid residue thus obtained is washed 2˜3 times with water to remove the solvent, the hydrolysis efficiency of the cellulose solid residue thus obtained can be greatly increased to 80˜90%. If the low-temperature drying is used according to the present invention, the enzymatic hydrolysis can be greater than 80%. Yet, the amount of water used is saved for about 10˜20 times of the weight of the cellulose solid residue, which dramatically reduces water used in production. The present invention improves the efficiency of lignocellulose saccharification; separates lignin, cellulose, and hemicellulose; and reduces water consumption during production with little water footprint.

To sum up, the present invention is a method of pretreating lignocellulose using solvent with little water footprint, where high-purity products of lignin, cellulose, and hemicellulose are separated for high-value applications; the present invention is applicable to high-price source material and lignocellulose containing high content of lignin; and the present invention effectively reduces water consumption during production for helping the plant domain lack of water resource.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method of using a solvent to pretreat lignocellulose with little water footprint, comprising steps of:
(a) providing a source material of lignocellulose,
wherein said source material of lignocellulose comprises cellulose, hemicellulose, and lignin; and has a lignin content more than 10 percents (%);
(b) cutting said source material of lignocellulose to desired sizes;
(c) uniformly mixing said source material of lignocellulose with a solvent through blending to process a high-temperature high-pressure reaction in a reactor,
wherein said solvent comprises an alcohol, water and an acid; and
wherein said high-temperature high-pressure reaction is processed at a temperature of 120˜160 celsius degrees (° C.) for 20˜120 minutes;
(d) separating solid content and liquid content in said reactor to obtain a cellulose solid residue and a hydrolyzate,
wherein said cellulose solid residue has a high content of cellulose and said hydrolyzate contains lignin and xylose;
(e) processing said hydrolyzate to precipitate lignin to be separated from xylose through a method selected from a group consisting of evaporation and solvent-concentration dilution;
(f) recycling said solvent;
(g) removing said solvent contained in said cellulose solid residue through low-temperature drying and recycling said solvent by looping back to step (f),
wherein said low-temperature drying is coordinated with air-suction and is processed at a temperature of 10˜50° C. under a pressure of 10˜150 millibars (mbar); and
(h) processing said cellulose solid through enzymatic hydrolysis with a cellulase,
wherein said hydrolysis has a saccharification rate up to 80%.

2. The method according to claim 1,
wherein said source material of lignocellulose comprises a material selected from a group consisting of wood chips and bagasse.

3. The method according to claim 1,
wherein said solvent has a concentration of an alcohol for 40˜75%, a concentration of water for 25˜60%, and a concentration of an acid for 0.5˜3%; said acid is selected from a group consisting of an inorganic acid and an organic acid; and a weight ratio of said solvent to said source material of lignocellulose is 4˜12.

4. The method according to claim 3,
wherein said inorganic acid is selected from a group consisting of sulfuric acid, hydrochloric acid and acetic acid.

5. The method according to claim 3,
wherein said alcohol is selected from a group consisting of methanol, ethanol, isopropanol, n-butanol and isobutanol.

6. The method according to claim 1,
wherein, in step (e), said solvent-concentration dilution is processed by adding a material selected from a group consisting of a solvent different from alcohol; and water.

7. The method according to claim 1,
wherein, in step (g), said drying is processed for a time of 0.5˜12 hours.

8. The method according to claim 1,
wherein said cellulose used in said hydrolysis has a density of 10˜30 filter paper units per gram (FPU/g) and is processed at a temperature of 45˜52° C. for a time of 24˜72 hours.

9. The method according to claim 1,
wherein, in step (d), a diluent is further added.

10. The method according to claim 9,
wherein said diluents is selected from a group consisting of water, an alcohol, an acid, and a combination thereof.
